(19) United States
(12) Patent Application Publication
(10) Pub. No.: US 2019/0134165 A1
(43) Pub. Date: May 9, 2019

(54) COMPOSITIONS AND METHODS FOR TREATING THYROID DISEASE

(71) Applicant: Script Essentials, LLC, Broomfield, CO (US)

(72) Inventor: Suzy Cohen, Broomfield, CO (US)

(21) Appl. No.: 16/237,933

(22) Filed: Jan. 2, 2019

Related U.S. Application Data
(62) Division of application No. 15/157,063, filed on May 17, 2016.
(60) Provisional application No. 62/163,820, filed on May 19, 2015.

(51) Int. Cl. A61K 38/48; A61K 31/198; A61K 9/48; A61K 36/704; A61K 33/24; A61K 33/32; A61K 33/30; A61K 33/04; A61K 33/18; A61K 31/714; A61K 31/4415; A23L 33/16; A23L 33/15; A61K 33/34; A61K 36/87; A23L 33/185; A23L 33/17; A23L 33/105; A23L 29/00

(52) U.S. Cl. CPC A61K 38/4813; C12Y 304/14005; A61K 9/4858; A61K 36/704; A61K 33/24; A61K 33/32; A61K 33/30; A61K 33/04; A61K 33/18; A61K 31/714; A61K 31/4415; A23L 33/16; A23L 33/15; A61K 33/34; A61K 36/87; A23L 33/185; A23L 33/17; A23L 33/105; A23L 29/06; A61K 9/4866; A61K 31/198

(57) ABSTRACT

The present disclosure relates to a compound and method of administering such compound for treating a patient suffering from thyroid disease and various thyroid-related disorders, including hyperthyroid and hypothyroid. The unique combination of the composition is preferably administered orally. The composition is preferably comprised of at least selenium, zinc, iodine, manganese, molybdenum, ashwaganda root powder, polygonum root extract, a digestive enzyme such as DPP-IV, L-tyrosine and mullein leaf, in pre-determined amounts. The composition can further comprise a palliative agent, and can be provided in the form of a capsule or tablet.

### CROSS REFERENCE TO RELATED APPLICATIONS

This application is a divisional of U.S. patent application Ser. No. 15/157,063, filed May 17, 2016, which claims priority and the benefit under 35 U.S.C. § 119(e) to U.S. Provisional Patent Application Ser. No. 62/163,820 filed May 19, 2015, which are incorporated herein in their entirety by reference.

### FIELD OF THE INVENTION

The present invention is directed to compounds, particularly dietary supplements, and methods for formulating and administering the same.

### BACKGROUND OF THE INVENTION

The human thyroid gland naturally produces the hormones thyroxine (T4) and triiodothyronine (T3), which are essential to the proper development and functions of cells in the human body. Thyroid hormones regulate the metabolism of fats, proteins and carbohydrates. In turn, the thyroid hormone affects how these compounds produce energy. In addition to these functions, thyroid hormones have several critical functions in the human body, especially pertaining to human metabolic and cardiovascular systems. For example, production of T4 supports cardiac output, a healthy heart rate, respiratory rate, and basal metabolic rate (BMR).

There are presently a number of known but unresolved problems relating to the human thyroid and in general thyroid disease and thyroid-related disorders. By way of example, a low BMR may cause a person to feel cold, fatigued and retain weight. Other problems are known to those of ordinary skill in the art. Therefore, it is desirable to provide a compound, such as a dietary supplement, that addresses these problems and otherwise improves upon the healthy function of the human thyroid gland.

According to studies, dietary supplements containing zinc were able to reestablish normal thyroid function in hypothyroid patients. In another study, nine of thirteen participants with low T3 (but normal T4) had mild to moderate zinc deficiency. After oral supplementation with zinc sulfate, the levels of T3 normalized, reverse T3 (rT3) decreased and TSH normalized. Therefore, it would be desirable to include zinc with a dietary supplement in order to positively influence human metabolism of thyroid hormones, and more specifically activation of T4 to T3.

In addition to the problems associated with unhealthy thyroid function, people with gastrointestinal diseases are also more susceptible to zinc deficiency. This deficiency can lead to thyroid dysfunction. Through experimentation it has been found that including zinc as a dietary supplement may be beneficial to people who: have conditions that affect the brain (epilepsy, schizophrenia, etc.); have an eating disorder (anorexia or bulimia); drink alcohol or coffee; fail to ovulate properly, or don't get a regular period; have sickle cell anemia; have Celiac disease or Irritable Bowel Syndrome; have kidney disease; or have diabetes.

More particularly, regular consumers of alcohol and alcoholics have difficulty retaining zinc in their diets, and are about 50% more likely to have a zinc deficiency due to their reduced ability to absorb nutrients. Furthermore, high amounts of zinc can cause a copper deficiency, which in turn can lead to a wide variety of health problems including excessive T4 in the bloodstream. This creates a situation where the human thyroid has to work harder, and may cause a person to feel clinically hypothyroid. Therefore, there is a need for a dietary supplement that promotes the health benefits of zinc and assists certain people to retain zinc in proper amounts to promote a health thyroid.

Selenium has also been found to be beneficial to treating autoimmune thyroid disease. Recent experimentation has shown that selenium supplementation reduced thyroid peroxidase (TPO) antibody levels in the blood, even in selenium sufficient patients. TPO antibodies attack the thyroid and cause autoimmune disease (Hashimoto's). Selenium can also significantly reduce inflammatory cytokines. Therefore, providing a composition that also comprises appropriate levels of selenium would also be beneficial. Other nutrients and compounds may provide further benefits to both the thyroid and adrenal glands of the human body, and are described in greater detail herein.

These problems and others are addressed by the compositions and methods described in detail below.

### SUMMARY OF THE INVENTION

In varying embodiments described herein, the invention relates to a compound that is capable of treating thyroid disease and various thyroid-related disorders. The unique combination of the composition is preferably administered orally in the form of a capsule. Methods for formulating the compound are also disclosed herein. The unique combination has synergistic advantages over previously known compositions. As disclosed in more detail in the detailed description, the present invention provides both compositions and methods for treating thyroid disease or thyroid-related disorders.

The composition is preferably comprised of a unique and novel formulation in pre-determined amounts, and further provides benefits previously unexpected. In addition to other health benefits described herein, the composition enhances the production of T4; provides for peripheral conversion of T4 to T3; assists the human body in optimizing metabolism; and is provided by way of a non-glandular formula, which comprises supportive nutrients for a healthy thyroid gland.

In certain embodiments, the composition provides additional nutrients necessary for conversion of T4 to T3 in tissues outside the human thyroid gland.

In a preferred embodiment, the composition is comprised of at least selenium, zinc, iodine, manganese, molybdenum, ashwaganda root powder, polygonum root extract, and a digestive enzyme component such as, by way of example but not limitation, DPP-IV, L-tyrosine and mullein leaf. Other ingredients may be included which promote healthy thyroid and adrenal glands of the human body as described in detail herein.

More particularly, a preferred embodiment of the present invention is a compound that comprises from about 0.5 to 3.5 mg of B6, from about 10 to 90 mcg of B12, from about 300 to 700 mcg of iodine, from about 50 to 250 mcg of selenium, from about 4 to 16 mg of zinc, from about 0.5 to 3.5 mg of manganese, from about 25 to 125 mcg of molybdenum, from about 300 to 600 mg of ashwaganda root powder, from about 50 to 170 mg of polygonum root extract, from about 700 to 1300 mg of a digestive enzyme (preferably DPP-IV), from about 40 to 110 mg of L-Tyrosine, and from about 20 to 80 mg of mullein leaf.

An aspect of the invention is a composition. The composition includes about 0.5 to 3.5 mg of B6, about 10 to 90 mcg of B12, about 300 to 700 mcg of iodine, about 50 to 250 mcg of selenium, about 4 to 16 mg of zinc, about 0.5 to 3.5 mg of manganese, about 25 to 125 mcg of molybdenum, about 300 to 600 mg of ashwagandha root powder, about 50 to 170 mg of an extract selected from the group consisting of polygonum root extract, a grape seed extract, or combinations thereof; and about 700 to 1300 mg of at least one digestive enzyme.

An aspect of the invention is a method for treating a thyroid disease. The method includes administering an effective amount of a composition to a patient suffering from the thyroid disease. The composition includes about 0.5 to 3.5 mg of B6, about 10 to 90 mcg of B12, about 300 to 700 mcg of iodine, about 50 to 250 mcg of selenium, about 4 to 16 mg of zinc, about 0.5 to 3.5 mg of manganese, about 25 to 125 mcg of molybdenum, about 300 to 600 mg of ashwagandha root powder, about 50 to 170 mg of an extract selected from the group consisting of polygonum root extract, a grape seed extract, or combinations thereof; and about 700 to 1300 mg of at least one digestive enzyme.

An aspect of the invention is a composition for treating thyroid disease. The composition includes B6, B12, iodine, selenium, zinc, manganese, molybdenum, ashwaganda root powder, an extract selected from the group consisting of polygonum root extract, a grape seed extract, or combinations thereof, and a digestive enzyme.

In one embodiment, the composition is provided as a dietary supplement. In one embodiment, the composition is administered in the form of a capsule. In an alternate embodiment, the composition is administered in the form of a tablet. In further embodiments, the composition comprises one or more palatability agents or otherwise has a flavor desirable for human consumption.

As used herein, the phrases “at least one”, “one or more”, and “and/or”, as used herein, are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Ranges will be discussed and used within the description of the invention. One skilled in the art would understand that any sub-range within the stated range would be suitable, as would any number within the broad range, without deviating from the invention.

Unless otherwise indicated, all numbers expressing quantities, dimensions, conditions, and so forth used in the specification and claims are to be understood as being modified in all instances by the term “about”.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein. It shall be understood that the term “means” as used herein shall be given its broadest possible interpretation in accordance with 35 U.S.C. Section 112(f). Accordingly, a claim incorporating the term “means” shall cover all structures, materials, or acts set forth herein, and all of the equivalents thereof. Further, the structures, materials, or acts and the equivalents thereof shall include all those described in the summary of the invention, brief description of the drawings, detailed description, abstract, and claims themselves.

These and other advantages will be apparent from the disclosure of the invention(s) contained herein. The above-described embodiments, objectives, and configurations are neither complete nor exhaustive. The summary of the invention is neither intended nor should it be construed as being representative of the full extent and scope of the present invention.

Moreover, references made herein to “the present invention” or aspects thereof should be understood to mean certain embodiments of the present invention and should not necessarily be construed as limiting all embodiments to a particular description. The present invention is set forth in various levels of detail in the summary of the invention and the detailed description, and no limitation as to the scope of the present invention is intended by either the inclusion or non-inclusion of elements in this summary of the invention. Additional aspects of the present invention will become more readily apparent from the detailed description.

### DETAILED DESCRIPTION

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

Composition:

An aspect of the present invention is directed to a composition. The composition can be used in treating thyroid disease, various thyroid-related disorders, or alcoholics or consumers of alcohol with difficulty retaining zinc, which is another aspect of the invention. The composition includes vitamin B6, vitamin B12, iodine, zinc, selenium, manganese, molybdenum, ashwagandha root powder, an extract, a digestive enzyme, L-thyrosine, and mullein leaf. The composition can include other materials.

Digestive Enzyme

In a preferred embodiment, the composition is comprised of at least one digestive enzyme. In a most preferred embodiment, the compound is comprised of a pre-determined amount of “Dipeptidyl peptidase 4” or DPP-IV. It has been found from experimentation that DPP-IV reduces absorption of gluten and casein, proteins found in wheat and dairy, respectively. As gluten and casein are known to attack and damage the thyroid glands of certain individuals, it is desirable to include a pre-determined amount of DPP-IV with the composition. In addition, certain individuals have a deficiency in this enzyme, and therefore have trouble breaking down the gluten protein. Partial digestion of the gluten protein can exasperate the gastrointestinal tract, inflame the small intestine, and provoke an immune response, which in turn attacks the human thyroid gland.

In a preferred embodiment, the composition comprises from about 700 to 1300 mg of a digestive enzyme, preferably DPP-IV. Other suitable enzymes include, but are not limited to, amylase, lipase, protease, proteolytic enzymes or other digestive enzymes, or combinations thereof. In a most preferred embodiment, the composition comprises about 1000 mg of DPP-IV. In alternate embodiments, one or more additional digestive enzymes are included. The DPP-IV can be in the form of protease DPP-IV.

Ashwagandha Root Powder

In a preferred embodiment, the composition is comprised of a pre-determined amount of the Ayurvedic botanical “ashwagandha,” which has been found through experimentation to improve T4 concentrations. Ashwagandha is also known as an adrenal adaptogen. When the adrenals go into a state of ‘overdrive’ they produce high amounts of cortisol. High cortisol levels can interfere with thyroid function, resulting in low T4 or poor conversion of T4 to the active form, T3. Ashwagandha has been found through experimentation to support normal levels of cortisol. In a preferred embodiment, the composition comprises from about 300 to 600 mg of ashwagandha root powder. In a most preferred embodiment, the composition comprises about 450 mg of ashwagandha root powder.

Selenium

In a preferred embodiment, the composition is comprised of a pre-determined amount of selenium. Selenium helps with thyroid hormone production and usage. More specifically, this antioxidant mineral is essential for the D (deiodinase) enzyme, which converts T4 to T3. Deficiency of selenium equates to lower T3 levels. In essence, deficiency in selenium could cause a person to be clinically hypothyroid, even though circulating levels of T3 and T4 appear to be normal. Selenium is also needed to balance excess thyroid activity that may be caused by external or internal stressors, and protects the thyroid gland from damage from excessive iodine exposure. In a preferred embodiment, the composition comprises from about 50 to 250 mcg of selenium. In a most preferred embodiment, the composition comprises about 150 mcg of selenium. The selenium can be provided as sodium selenite.

Iodine

In a preferred embodiment, the composition further comprises a pre-determined amount of iodine. Iodine has been found through experimentation to assist in the formation of T4, which in turn promotes healthy thyroid function. In a preferred embodiment, the composition comprises from about 300 to 700 mcg of iodine. In a most preferred embodiment, the composition comprises about 500 mcg of iodine. The iodine can be provided as potassium iodide.

L-Tyrosine

In a preferred embodiment, the composition further comprises a pre-determined amount of L-tyrosine. The amino acid L-tyrosine has been discovered through experimentation to be a desirable component of both T4 and T3 formation. In a preferred embodiment, the composition comprises from about 40 to 110 mg of L-Tyrosine. In a most preferred embodiment, the composition comprises about 75 mg of L-Tyrosine.

Polygonum Root Extract

In a preferred embodiment, the composition further comprises a pre-determined amount of polygonum root extract and/or grape seed extract. Through experimentation it has been discovered that conversion of T4 to T3 may be inhibited by heavy metal toxicity and lipid peroxidation. It is believed that dosages of supplements containing polygonum root extract and/or grape seed extract, such as with the antioxidant blend “Oxyphyte,” can counterbalance this inhibition. Polygonum root has also been found to activate SIRT1 (silent information regulator 1), dubbed the “skinny gene.” With respect to thyroid function, polygonum root may help prevent or slow growth of both papillary and follicular thyroid cancer, and further enhances iodine utilization, metabolism and thyroid function by affecting SIRT1 gene and other genes. Thus, in a preferred embodiment, the composition comprises from about 50 to 170 mg of polygonum root extract and/or from about 150 to 300 mg of grape seed extract. In a most preferred embodiment, the composition comprises about 110 mg of polygonum root extract and/or about 200 mg of grape seed extract.

B6

In a preferred embodiment, the composition further comprises a pre-determined amount of B6. Pyridoxal 5′ Phosphate (P5P) is an active form of vitamin B6. It has been discovered through experimentation that a deficiency of this B vitamin can make an immune system more likely to attack the thyroid. In addition, P5P is required to work with the mineral zinc, which is necessary to make the human thyroid hormone function, and some individuals cannot readily convert B6 to its biologically active (usable) form. Low levels of B6 are also associated with premenstrual difficulties, and a deficiency of P5P can lead to hypothyroidism and autoimmune thyroid disease. In a preferred embodiment, the composition comprises from about 0.5 to 3.5 mg of B6. In a most preferred embodiment, the composition comprises about 2 mg of B6.

B12

In a preferred embodiment, the composition further comprises a pre-determined amount of the vitamin B12, because through experimentation it has been determined that insufficiency of B12 interferes with the activity of thyroid hormones. Methylcobalamin is one of the active forms of vitamin B12. Methylcobalamin and B12 have been found to play a key role in cell metabolism of the human body, increasing energy, improving mental sharpness, and promoting healthy nervous system function. Research indicates that nearly 40% of hypothyroid patients suffer B12 deficiency. In a preferred embodiment, the composition comprises from about 10 to 90 mcg of B12. In a most preferred embodiment, the composition comprises about 50 mcg of B12.

Manganese

In a preferred embodiment, the composition further comprises a pre-determined amount of manganese. Manganese is believed to promote healthy mitochondria, which convert glucose into energy by spawning ATP. It has been found that the thyroid hormone does not work unless mitochondria are alive and functioning. As it pertains to thyroid health, this enzyme (which is dependent on manganese) is the primary antioxidant and protector of thyroid hormone in the human liver. Furthermore, most T4 and T3 conversion occurs in cells from the liver. Additionally, manganese helps people who are “pear-shaped” improve metabolism, and reduce fat accumulation in the liver, which can cause lipid peroxides. In a preferred embodiment, the composition comprises from about 0.5 to 3.5 mg of manganese. In a most preferred embodiment, the composition comprises about 2 mg of manganese. The manganese can be provided as manganese gluconate.

Molybdenum

In a preferred embodiment, the composition further comprises a pre-determined amount of molybdenum. The human body requires molybdenum to convert sulfites in meats, dried fruit, wine and sulfur-containing foods (for example, broccoli, garlic, or onions) into sulfate. Certain individuals have a genetic mutation or single nucleotide polymorphism (“snp”—pronounced snip), which causes difficulty in making this conversion. The enzyme that makes this conversion is called “sulfite oxidase.” Individuals who suffer from poor mental focus or lack of concentration may have molybdenum deficiencies. Therefore, in a preferred embodiment, the composition comprises from about 25 to 125 mcg of molybdenum. In a most preferred embodiment, the composition comprises about 75 mcg of molybdenum. The molybdenum can be provided as sodium molybdate in some embodiments.

Zinc

Zinc aids in the conversion of T4 to T3. In a preferred embodiment, the composition comprises from about 5 mg to about 50 mg of zinc. In a most preferred embodiment, the composition comprises about 10 mg of zinc. The zinc can be provided as zinc M nomethionine.

Mullein Leaf

Mullein leaf is an herb is known botanically as “Berbascum Thapsus” and has strong medicinal benefits and antibiotic actions against tuberculosis, leprosy and many other pathogens.

Furthermore, mullein possesses strong anti-inflammatory, anti-tumor, antifungal, antibacterial and anti-viral actions. It also has analgesic properties from its healing polysaccharides and flavonoids, which reduce inflammatory cytokines (by blocking NF-kB, a pathway in the body that churns pain-causing compounds). Because of its ability to reduce superoxide free radicals, mullein leaf may also reduce glandular inflammation associated with Graves' disease or Hashimoto's. In a preferred embodiment, the composition comprises from about 20 to 80 mg of mullein leaf In a most preferred embodiment, the composition comprises about 50 mg of mullein leaf.

Optional Components

In varying embodiments, the composition can further comprise one or more of calcium phosphate, hydroxypropyl methylcellulose, and copper gluconate. In addition, the composition of a preferred embodiment is substantially free of gluten, wheat, egg, peanuts, tree nuts, dairy, sugar, corn, soy, artificial colors, preservatives, fish and shellfish.

In addition to the benefits described herein, several of the botanicals and nutrients described in varying embodiments of the present invention have been found to exhibit antifungal and antibacterial activity against a wide variety of organisms, which in turn provides an additional layer of protection to the human thyroid gland.

According to certain embodiments, the compositions described herein can further be provided with one or more palatability agents. These palatability agents serve to add flavor to the composition so that an effective dosage is easier to be ingested. It is within the scope of the present invention that any safe, flavor enhancing palatability agent can be used in a composition of the present invention. Particularly suitable palatability agents for use in the composition of the present invention include, but are not limited to, plant oils, plant hydrolysates, yeast, and yeast hydrolysates.

An aspect of the invention is a method to treat a thyroid disease with a composition comprising vitamin B6, vitamin B12, iodine, zinc, selenium, manganese, molybdenum, ashwagandha root powder, an extract, and a digestive enzyme. The composition can include other components such as L-thyrosine, and mullein leaf, calcium phosphate, hydroxypropyl methylcellulose, copper gluconate, and combinations thereof. The patient is treated by providing an effective amount of the composition to affect the thyroid. By way of example, between 1 and 3 capsules of the composition can be provided to a patient per day, in some embodiments 2 capsules per day. The capsules can be taken by the patient daily, preferably in the morning before any other food is ingested by the patient. In some embodiments, the dosage can be adjusted to result in an adequate thyroid level in the patient.

An aspect of the invention is a method to treat a patient with a zinc deficiency. The method includes administering a dose of a composition comprising vitamin B6, vitamin B12, iodine, zinc, selenium, manganese, molybdenum, ashwagandha root powder, an extract, and a digestive enzyme to the patient suffering from the zinc deficiency. The composition can include other components such as L-thyrosine, and mullein leaf, calcium phosphate, hydroxypropyl methylcellulose, copper gluconate, and combinations thereof. The dose can be daily, every other day, every week, or as necessary to provide affect the zinc deficiency in the patent. By way of example, between 1 and 3 capsules of the composition can be provided to a patient per day, in some embodiments 2 capsules per day. The capsules can be taken by the patient daily, preferably in the morning before any other food is ingested by the patient. In some embodiments, the dosage can be adjusted to result in an adequate zinc level in the patient.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims. For further illustration, the book “Thyroid Healthy, Lose Weight, Look Beautiful and Live the Life You Imagine” authored by the named inventor of the present invention is expressly made a part of this disclosure and incorporated by reference herein in their entirety.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

What is claimed is:

1. A method for treating a thyroid disease comprising: administering an effective amount of a composition to a patient suffering from the thyroid disease, wherein the composition comprises:
0.5 to 3.5 mg of B6;
10 to 100 mcg, of B12;
300 to 700 mcg of iodine;
50 to 250 mcg of selenium;
4 to 16 mg of zinc;
0.5 to 3.5 of manganese;
25 to 125 mcg to molybdenum;
300 to 600 mg of ashwaganda root powder;
50 to 170 mg of an extract selected from the group consisting of polygonum root extract, a grape seed extract, or combinations thereof; and
100 to 800 mg of at least one digestive enzyme.

2. The method of claim 1, wherein the thyroid disease is selected from the group consisting of hyperthyroid, and hypothyroid.

3. The method of claim 1, wherein the composition is administered orally in the form of a capsule.

4. The method of claim 3, wherein the method further comprises administering between 1 and 3 capsules of the composition to the patient per day.

5. The method of claim 1, wherein the composition administered to the patient further comprises 40 to 110 mg of L-Tyrosine.

6. The method of claim 5, wherein the composition administered to the patient further comprises 20 to 80 mg of mullein leaf.

7. The method of claim 6, wherein the digestive enzyme is DPP-IV.

8. A method for treating a thyroid disease by administering to a patient in need thereof a composition, wherein the composition comprises:
about 2 mg of B6;
about 90 mcg of B12;
about 500 mcg of iodine;
about 150 mcg of selenium;
about 10 mg of zinc;
about 2 mg of manganese;
about 75 mcg of molybdenum;
about 450 mg of ashwagandha root powder;
about 102 mg of polygonum root extract;
about 200 mg of DPP-IV;
about 75 mg of L-Tyrosine; and
about 50 mg of mullein leaf

9. The method of claim 8, wherein the B12 administered to the patient is in the form of methylcobalamin.

10. The method of claim 8, wherein the composition administered to the patient further comprises calcium phosphate.

11. The method of claim 8, wherein the composition administered to the patient further comprises copper gluconate.

12. The method of claim 8, wherein the composition administered to the patient further comprises hydroxypropyl methylcellulose.

13. The method of claim 8, wherein the composition administered to the patient does not comprise additives selected from the group consisting of gluten, wheat, egg, peanuts, tree nuts, dairy, sugar, corn, soy, artificial colors, preservatives, fish and shellfish.

14. The method of claim 8, wherein the composition administered to the patient further comprises at least one palatability agent.

15. The method of claim 14, wherein the palatability agent is selected from the group consisting of plant oils, plant hydrolysates, yeast, and yeast hydrolysates.

16. The method of claim 15, wherein the thyroid disease is selected from the group consisting of hyperthyroid, and hypothyroid.

17. The method of claim 16, wherein the composition is administered orally in the form of a capsule.

18. The method of claim 17, wherein the method further comprises administering between 1 and 3 capsules of the composition to the patient per day.

19. The method of claim 16, wherein the composition is administered to the patient as a dietary supplement.
